(19) United States
(12) Patent Application Publication
(10) Pub. No.: US 2025/0018145 A1
(43) Pub. Date: Jan. 16, 2025

(54) DEEP BRAIN SOUND STIMULATION (DBSS) FOR TARGETED BRAINWAVE ENTRAINMENT AND NEUROMODULATION

(71) Applicant: Ugur Dogan, The Hague (NL)

(72) Inventor: Ugur Dogan, The Hague (NL)

(21) Appl. No.: 18/766,470

(22) Filed: Jul. 8, 2024

Related U.S. Application Data
(63) Continuation-in-part of application No. 15/585,313, filed on May 3, 2017, now abandoned.

(51) Int. Cl. A61M 21/02; A61M 21/00

(52) U.S. Cl. CPC A61M 21/02; A61M 2021/0027

(57) ABSTRACT

This disclosure pertains to methods and systems for brainwave entrainment and neuromodulation utilizing audio signals, specifically through Deep Brain Sound Stimulation (DBSS). DBSS employs audio frequencies to target and modulate brain regions, offering therapeutic benefits in areas such as stress reduction, focus enhancement, cognitive function improvement, and the treatment of neurological disorders. The system generates audio tones tailored with a brainwave protocol that includes primary and secondary frequency numbers. These tones are finely modulated to produce specific brainwave patterns that facilitate targeted neuromodulation. This technology delivers customized stimulation based on individual neurological profiles, enhancing both the effectiveness and safety of the therapy. DBSS represents a significant advancement over traditional brainwave entrainment methods by providing a non-invasive, user-friendly, and efficient way to potentially improve mental health and cognitive abilities.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A “SEQUENCE LISTING” (IF APPLICABLE)

Not applicable.

### BACKGROUND OF THE INVENTION

Brainwave entrainment involves synchronizing brainwave frequencies with external rhythmic stimuli, such as sound. This technique has potential applications in treating various neurological and psychological conditions. Traditional methods often lack the precision to target specific brain regions effectively. Deep Brain Sound Stimulation (DBSS) addresses this limitation by utilizing precisely modulated audio signals to stimulate specific brain regions, thereby enhancing the effectiveness of brainwave entrainment.

Unlike traditional brainwave entrainment methods, DBSS offers an unprecedented range of treatments encompassing over 130 specific disorders across various categories including mood, personality, behavioral, trauma-related, psychotic, neurological, and musculoskeletal disorders, among others. This extensive scope is made possible by our advanced technology's ability to precisely target and modulate brainwaves specific to each disorder, a capability not found in conventional brainwave entrainment technologies.

Moreover, since its introduction in 2015, DBSS has been widely accepted, as demonstrated by over 12,000 therapies sold with a remarkable satisfaction rate of 99.5%, reflecting minimal refund requests of just 0.5%. This data not only shows significant commercial success but also user trust and satisfaction, positioning DBSS as a leader in non-invasive brain stimulation technologies.

Distinction from Traditional Brainwave Entrainment Methods:

Traditional brainwave entrainment techniques, such as binaural beats, monaural beats, and isochronic tones, typically involve the use of general frequency ranges corresponding to different brainwave states (e.g., alpha, beta, theta, delta, and gamma). These methods generally lack the precision to target specific brain regions effectively and do not offer the detailed fine-tuning capabilities necessary for precise neuromodulation.
Binaural Beats: Auditory processing artifacts created when two tones of slightly different frequencies are presented separately to each ear, resulting in the perception of a third tone, which is the mathematical difference between the two frequencies.
Monaural Beats: Simple beats created by combining two tones of slightly different frequencies, played in the same ear, resulting in the perception of a beat frequency that is the difference between the two frequencies.
Isochronic Tones: Regular beats of a single tone, turned on and off at regular intervals, known for their simplicity and effectiveness in brainwave entrainment.

In contrast, the Deep Brain Sound Stimulation (DBSS) method employs a highly specific frequency mapping system, utilizing primary and secondary frequency numbers to target precise brain regions.
Primary Frequencies: Correspond to major brain regions (e.g., 30 Hz for the brainstem, 60 Hz for the frontal cortex).
Secondary Frequencies: For instance, within the frontal cortex (primary frequency 60), secondary frequencies range from 61 to 250, allowing detailed targeting of subregions, starting from the motor cortex (secondary frequency 61) to the orbitofrontal cortex (secondary frequency around 250).

This approach allows for precision targeting by using secondary frequency numbers and their decimal values to finely tune the stimulation within specific brain regions. For example, if 250 represents a physical location with a size of 1 square mm, then within this location, secondary frequencies can be adjusted with decimal precision, such as from 250.0 to 250.9. This level of granularity helps to ensure highly targeted neuromodulation.

The DBSS method creates a mix of isochronic and monaural tones, specifically modulated to activate targeted brain areas. Proprietary modulation settings are derived from extensive research and are crucial for the efficacy of the DBSS protocol. Moreover, DBSS sessions are structured based on scientific research and are designed to stimulate multiple brain locations simultaneously, ensuring comprehensive therapeutic outcomes. This structured approach allows for an unmatched level of targeting precision, supporting complex and nuanced therapeutic strategies that significantly surpass the capabilities of traditional methods.
Note: The frequency numbers used in this description are illustrative and not the actual frequencies used in the DBSS protocol, which are proprietary and withheld to safeguard intellectual property.
   Additional Comparative Analysis with Other Brain Stimulation Techniques:

Traditional methods of brain stimulation, such as Electroencephalogram (EEG) Neurofeedback, Transcranial Magnetic Stimulation (TMS), and Transcranial Direct Current Stimulation (tDCS), have provided various degrees of therapeutic benefit. TMS utilizes magnetic fields to generate electric currents in the brain, typically targeting depression and anxiety. tDCS employs direct electrical currents for cognitive enhancement and mood stabilization.

When comparing DBSS to these non-invasive brain stimulation and modulation techniques, it is essential to recognize that DBSS builds on the fundamental principles of brainwave entrainment but aims to offer greater precision and efficacy. Unlike TMS and tDCS, which often produce broad and imprecise effects, DBSS provides fine-tuned, specific targeting of brain regions. This allows for a higher degree of control over brainwave modulation, critical for effective treatment of complex medical and psychological conditions. DBSS's advanced targeting capability dramatically enhances the potential for therapeutic interventions, making it a significant advancement over existing technologies.

### BRIEF SUMMARY OF THE INVENTION

The present invention relates to a method and system for inducing brainwave entrainment and neuromodulation using audio signals. The method involves generating specific audio tones, applying a brainwave protocol consisting of primary and secondary frequency numbers, modulating the audio signals to create a mix of isochronic and monaural tones, and outputting the modulated signals to achieve desired brainwave frequencies and stimulate targeted brain areas. When listened to with one ear, the tones activate only one side of the brain. This targeted approach aims to improve therapeutic outcomes and enhance neural synchronization and neuroplasticity.

### BRIEF DESCRIPTION OF DRAWINGS

FIG. 1: Overview of the DBSS System Illustrates all major components, including the audio source, modulation module, and output device. This provides a high-level view of the system architecture and its main components.

FIG. 2: Detailed Flowchart of the Modulation Process Shows each step from generating the tone to outputting the modulated signals, emphasizing the modulation techniques used to create a mix of isochronic and monaural tones. This flowchart ensures clarity on the procedural steps and technological specifics of the modulation process.

FIG. 3: Circuit Diagram of Audio Signal Modulation Details the hardware setup for the signal processing unit. This diagram is essential for understanding the electronic components and their interconnections required for the modulation process.

FIG. 4: Software Interface Screenshots Demonstrates the user interface for generating tones and applying modulation parameters, essential for replicating the invention. This provides insight into the software configuration and user interaction necessary for the DBSS system.

FIG. 5: Frequency Mapping Table Compares traditional brainwave entrainment methods (alpha, beta, theta, delta, gamma) with DBSS frequency mappings (primary and secondary numbers for specific brain regions). This table highlights the differences and unique capabilities of DBSS, showing how it targets specific brain areas.

FIG. 6: Frequency Mapping Diagram Illustrates how primary and secondary frequency numbers correspond to specific brain regions. For example, primary frequency X might correspond to the frontal cortex, and secondary frequencies fine-tune the specific location Y within that region. This visual representation aids in understanding the precise targeting mechanism of DBSS.

FIG. 7: Precision Targeting through Decimal Levels Shows how the secondary frequency numbers can be adjusted to decimal levels for precise targeting within a brain region, breaking down frequencies into more detailed increments for specific neuromodulation. This figure is crucial for demonstrating the granularity and precision achievable with DBSS, potentially down to individual neurons.

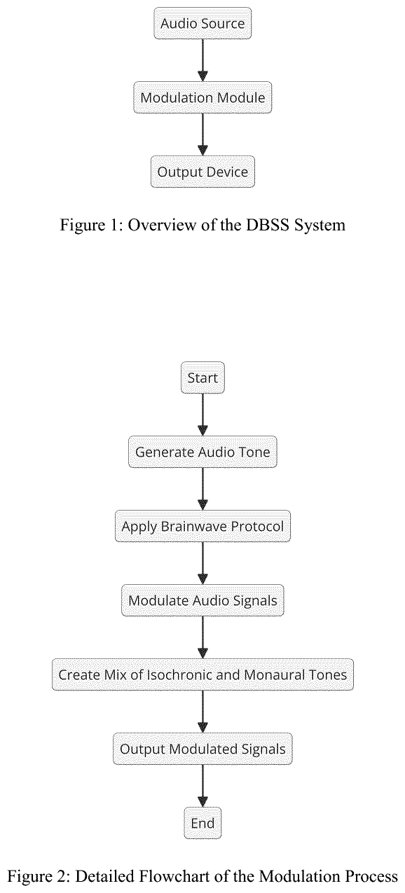

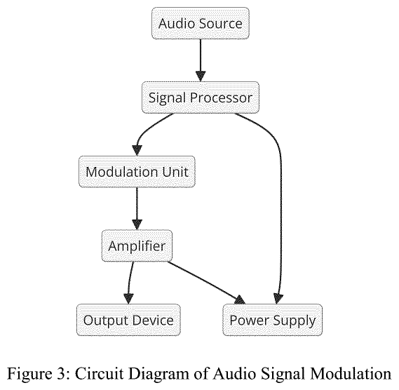

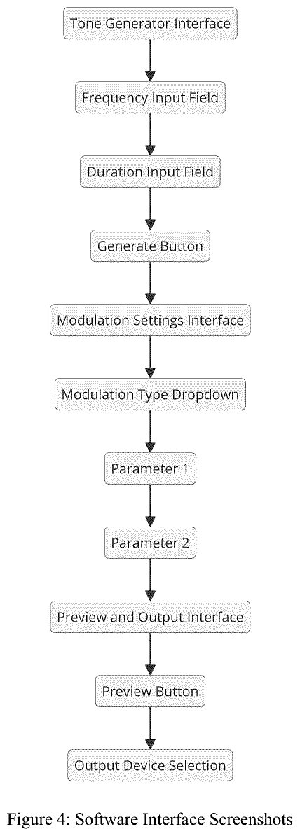

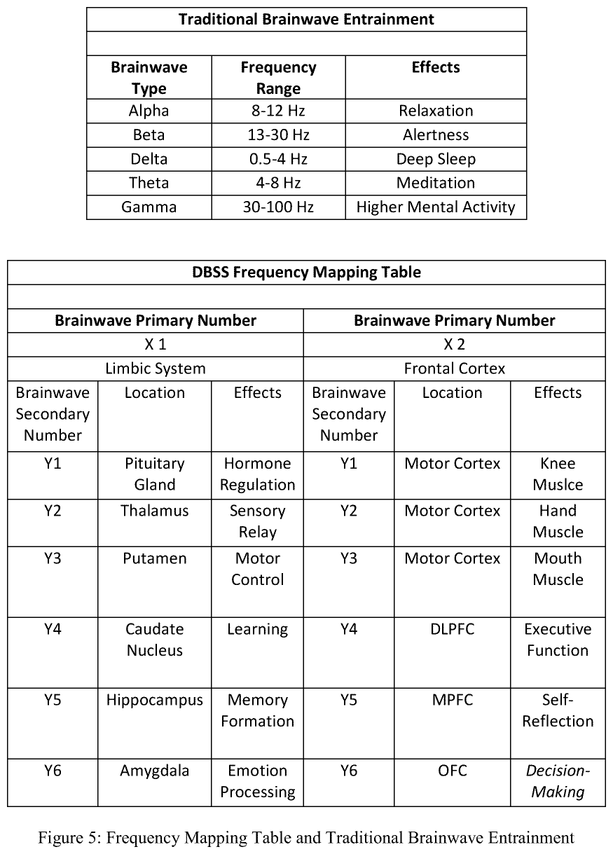

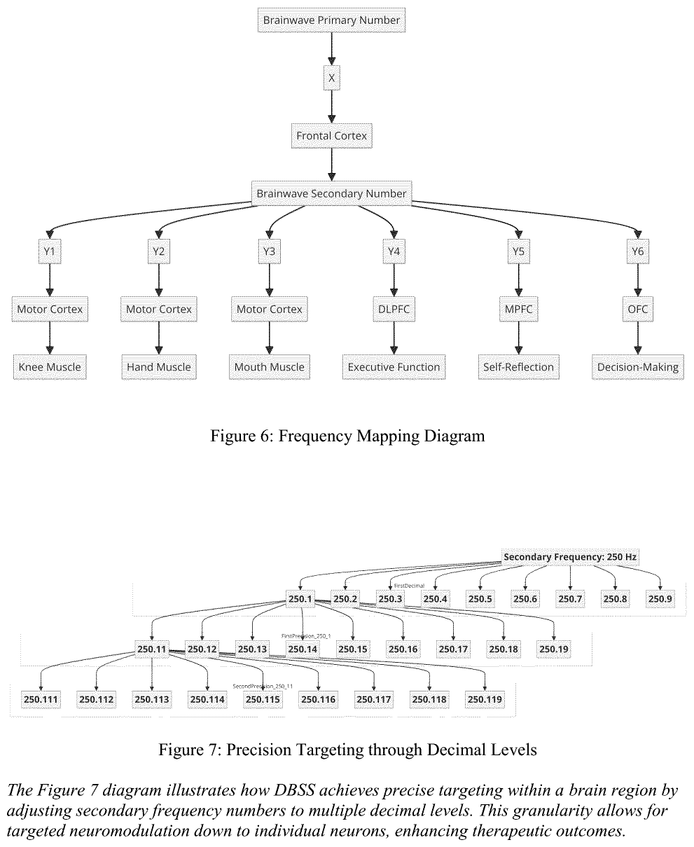

### DETAILED DESCRIPTION OF THE INVENTION: TERMINOLOGY

Brainwave Protocol: A predefined set of parameters used to modulate audio signals to induce specific brainwave frequencies.

Primary Frequency Numbers: Primary frequency values targeted in the brainwave protocol.

Secondary Frequency Numbers: Frequency values related to the primary frequency, used to fine-tune the targeting within a brain region.

Modulation Parameters: Variables controlling how audio signals are altered to achieve the desired brainwave frequencies.

### Method Overview:

The invention encompasses a method for inducing brainwave entrainment using audio signals modulated according to a brainwave protocol. The protocol includes primary and secondary frequency numbers and modulation parameters. The audio signals are generated using Audacity software, modulated using specific plugins, and output through headphones or speakers to target specific brain regions for therapeutic effects.

### Step-by-Step Process:

### Step 1: Generating Audio Tones

Description: Use Audacity software to generate specific audio tones.

### Technical Details:

Software: Audacity.
Tone Generation: Use the “Generate>Tone” menu in Audacity to create a sine waveform tone.
Frequency Selection: Choose a specific frequency number based on the brain region to be stimulated. The amplitude is variable and not crucial for the process.
Duration: Set the duration to a minimum of 3 minutes to ensure effective brainwave entrainment. The first 2 minutes level up to the entraining level, and the last 1 minute maintains continuous entrainment, allowing the stimulated activity to stay active for up to 24 hours or slowly fade back to normal.

### Step 2: Modulating the Audio Tones

Description: Apply modulation to the generated tones to create a mix of isochronic and monaural brainwave tones.

### Technical Details:

Modulation Plugin: Use a modulation plugin in Audacity to modulate the tone. This step is crucial as it ensures the brain processes the sound as a brainwave entrainment signal.
Proprietary Modulation Settings: Specific modulation settings are proprietary and withheld to safeguard intellectual property. These settings are derived from extensive research and mapping of brain regions.
Monoaural and Isochronic Mix: The modulation creates a combination of isochronic and monaural tones. Listening with one ear results in the activation of only one side of the brain, enhancing targeted stimulation.

### Step 3: Applying the Brainwave Protocol

Description: Use the brainwave protocol to determine the combination of primary and secondary frequency numbers.

### Technical Details:

Frequency Mapping: The primary frequency numbers correspond to specific brain regions. For example, frequencies might correspond to the brainstem, limbic system, temporal cortex, frontal cortex, parietal cortex, occipital cortex, and cerebellum. The secondary frequency numbers provide more specific locations within these regions.
Excel Mapping: A detailed map in an Excel sheet indicates which combination of frequency numbers to use for stimulating specific brain locations. This map was developed through extensive testing and validation involving sensory feedback and functional effects. This precision allows for the application of DBSS across a wide range of disorders, making it a versatile tool in both psychological and physical therapeutic contexts.

### Step 4: Outputting the Modulated Signals

Description: Deliver the modulated audio signals to the user.

### Technical Details:

Output Device: Use high-fidelity headphones or speakers to ensure the integrity of the modulated signals.
Listening Method: Standard listening is done through both ears, which activates both sides of the brain. An additional feature of DBSS is that listening with one ear can stimulate only one side of the brain, allowing for more precise targeting of brain regions.
Volume Adjustment: Adjust the volume to a comfortable level to ensure an effective and pleasant listening experience.

### Additional Technical Specifications:

### Precision and Decimal Levels:

Precision Targeting: The DBSS protocol excels in precision targeting by fine-tuning secondary frequency numbers to various decimal levels, depending on the specificity required for different brain systems. For example, at a base frequency like 75 Hz, which corresponds to a designated area of the brain, the granularity of our system allows division into finer segments, adaptable to the needs of the specific target region. Frequencies can be extended to one, two, three, or even four decimal places as necessary, with the potential to further refine targeting to a fifth decimal place, such as from 75.00000 to 75.99999, when extreme precision is needed.
This variable level of precision allows DBSS to tailor its approach to the complexity of the brain structure being targeted. In scenarios where the system permits, targeting can be refined to address up to 1,000,000 distinct locations within a very small area by utilizing the full five-decimal precision. However, the specific decimal level utilized varies depending on the required resolution for effective neuromodulation in that region. This flexibility in frequency adjustment enables highly targeted neuromodulation, which far exceeds the capabilities of traditional brainwave entrainment methods.
The ability to perform such ultra-fine segmentation allows for the customization and specificity necessary for targeting complex neural pathways and achieving nuanced therapeutic effects. This exceptional precision supports interventions that are tailored to the minutest differences within very small regions, facilitating highly effective and individualized treatment outcomes.

### Regulatory Considerations and Compliance:

The deployment of Deep Brain Sound Stimulation (DBSS) via mobile applications incorporates rigorous adherence to app store regulations, which mandate stringent review processes for health-related applications. Since its introduction in 2015, DBSS has maintained a significant presence in major app stores, reflecting its compliance with these regulations. Over 12,000 units of the DBSS app have been sold, with more than 3 million user interactions recorded to date. The absence of significant safety complaints and the sustained availability of the DBSS app in these regulated platforms substantiate the safety and regulatory compliance of the technology. This extensive user base and the continuous vetting by app stores such as Google Play and Apple App Store underscore the robustness of DBSS against the stringent safety and effectiveness standards required for health applications.

### Safety, Effectiveness, and User Satisfaction:

DBSS technology has demonstrated a remarkable safety profile over its operational history. The technology has engaged over half a million users with no major safety issues reported, underpinning its reliability and safety. The extensive user feedback collected through the app underscores a high satisfaction rate, with minimal refunds or complaints, highlighting both the effectiveness and user acceptability of the treatment. The proactive monitoring by app stores, coupled with our internal safety assessments, ensures that DBSS continues to meet high safety standards. This ongoing verification process aligns with the best practices for health applications as outlined by major tech platforms.

### Ethical Considerations:

In alignment with industry standards for digital health technologies, DBSS adheres to strict ethical guidelines concerning user consent and data privacy. The application's operations are in full compliance with the privacy policies mandated by app stores, which include detailed user agreements that ensure informed consent. These policies are meticulously designed to protect user data and provide transparency regarding the use and protection of personal information. By adhering to these guidelines, DBSS ensures that ethical considerations are prioritized, enhancing user trust and safeguarding participant welfare.

### Additional Information for Validation:

Identifying Brain Regions: Extensive testing with various brainwave frequencies and settings achieved a significant milestone in 2014, identifying the dorsolateral prefrontal cortex (DLPFC) through sensations of activity and functional effects. This breakthrough enabled detailed mapping of brain regions, with primary frequencies corresponding to specific brain regions and secondary numbers specifying exact locations within those regions (Heilbron et al., 2018; Zilles et al., 2018).

Validation Through Sensory Feedback: Sensory feedback is utilized akin to established medical practices, relying on patient-reported sensations for accurate diagnosis and treatment (Breivik et al., 2008; Schaefer et al., 2022).

Scientific Support: Literature supports sensory stimulation in neuroimaging and brain mapping, utilizing patient responses to map brain activity and validate stimulation efficacy (Logothetis et al., 2008; Glover et al., 2022).

Neural System Mapping and Body Location Targeting: Scientific evidence supports that specific brain regions, including those involved in hormonal regulation such as the hypothalamus, pituitary gland, and pineal gland, have defined mapping structures that can influence various body parts. These structures regulate crucial hormones like vasopressin, growth hormone, and melatonin, impacting specific areas of the body through well-defined feedback loops and mapping structures. Deep Brain Sound Stimulation (DBSS) technology leverages these mappings for more than hormonal regulation, extending its applications to over 40 physical conditions across categories such as Musculoskeletal Issues, Cardiovascular and Respiratory Disorders, Digestive and Metabolic Disorders, Cancer and Tumor Management, Sensory Disorders, and Other Physical Conditions. These treatments use neural systems to stimulate specific physical body locations, with a reported average satisfaction rate of 99.5%, underscoring their effectiveness. Detailed efficacy rates for each treatment are available on the Vital Tones website.

### Supporting Scientific Research:

TMS (Transcranial Magnetic Stimulation) aids in stroke rehabilitation by improving motor recovery functions (Lefaucheur et al., 2020).
DBS (Deep Brain Stimulation) improves motor functions in Parkinson's disease (Volkmann et al., 2010).
VNS (Vagus Nerve Stimulation) modulates autonomic functions such as heart rate and gastrointestinal motility (De Ferrari et al., 2011).

The results and effectiveness of these DBSS treatments continue to be validated through ongoing research, with several clinical studies underway to further substantiate these claims. This rigorous approach aims to ensure that DBSS applications in physical health are scientifically substantiated.

Ongoing Research: Additional manuscripts detailing the application of DBSS in brainwave therapy are under review, further demonstrating the ongoing research and validation efforts in this field. These manuscripts include:
“Advancing Brainwave Therapy: Precision Stimulation with Deep Brain Sound Technology”
“Advancing Brainwave Therapy for Borderline Personality Disorder: Precision Stimulation with Deep Brain Sound Technology”

Furthermore, we encourage reviewers to consult our Vital Tones website for the most recent publications and ongoing research supporting DBSS. This site continuously updates with new findings and peer-reviewed articles that validate and elaborate on the efficacy and safety of DBSS, providing a comprehensive resource for assessing the substantial advancements our technology contributes to the field.

Investigating Mechanisms of Action in DBSS: The mechanisms through which DBSS influences brain function are currently under rigorous investigation. Proposed mechanisms include:
Synaptic Reconnection and Neuroplasticity: It is hypothesized that DBSS can promote synaptic reconnections and enhance neural plasticity, aligning with observed neural growth patterns (McEwen et al., 2016, and Anacker et al., 2017).
Frequency-Specific Stimulation and Synaptic Plasticity: Preliminary research supports the hypothesis that DBSS enhances synaptic plasticity by targeting neural pathways with specific frequency pairs (Jones et al., 2007).
Sensory-Induced Neural Activation: DBSS might activate neural circuits by matching sound frequencies with brain tissue resonance frequencies, potentially enhancing cognitive and sensory processing (Draganski et al., 2006).
Modulation of Neurotransmitter Release: Ongoing investigations explore DBSS's potential to modulate key neurotransmitters like serotonin, dopamine, and oxytocin, crucial for brain function and well-being (Koo et al., 2019).

Scientific evidence has robustly demonstrated the efficacy of brainwave entrainment in modulating brain states, setting a foundation for advanced technologies like DBSS. Alpha wave entrainment, for instance, significantly enhances relaxation and reduces anxiety, while beta wave entrainment has been shown to improve focus and cognitive performance. These findings underpin the DBSS technology, providing a scientific basis for its enhanced precision and effectiveness in brainwave modulation for therapeutic purposes. The progression from traditional methods to DBSS illustrates significant advances in the specificity and potential impact of brainwave entrainment.

Studies on brainwave entrainment using auditory stimuli have shown promising results in various therapeutic areas:
Perceptual Relevance of Brain Oscillations: Research by Thut et al. (2011) highlights how non-invasive rhythmic auditory stimulation can entrain brain oscillations, which are crucial for perception and cognitive processes.
Auditory Beat Perception and Cognitive Functions: Fujioka et al. (2015) explored how beta-band oscillations, triggered by auditory beats, play a role in the hierarchical organization of temporal prediction and auditory perception, enhancing cognitive functions such as focus and memory.
Multi-Sensory Gamma Stimulation in Alzheimer's Disease: Martorell et al. (2019) demonstrated that gamma frequency auditory stimulation could ameliorate Alzheimer's-associated pathology and significantly improve cognitive functions, indicating the potential therapeutic effects of sound-based brainwave entrainment in neurodegenerative diseases.

These cases underscore the effectiveness of sound-based brainwave entrainment in enhancing cognitive functions and managing neurological conditions, supporting the potential application and efficacy of technologies like DBSS.

The case of a 24-year-old male with a porencephalic cyst and right-sided hemiparesis provides a detailed example of DBSS application. This case report is authored by myself and serves as a demonstration of the DBSS protocol's efficacy and application in clinical settings. The patient underwent multiple DBSS sessions targeting specific brain areas, including the lateral ventricles, thalamus, motor cortex, cerebellum, and basal ganglia. The treatment led to significant improvements in motor control and reduction of the cyst size from 2.6 cm to 8 mm over a year. Specific frequencies and protocols were tailored to target the affected brain areas, resulting in measurable clinical improvements, as evidenced by MRI scans and neurological evaluations. The outcomes included increased muscle power, improved coordination, and enhanced bone density. Validation of the DBSS protocol was achieved through systematic collection and analysis of sensory feedback during and after each session. The patient documented improvements in muscle stiffness, motor control, and overall physical skills, which were crucial in refining the treatment protocols. Objective measures such as periodic MRI scans, DEXA scans, and blood tests further supported the treatment's efficacy, corroborating the sensory feedback data. Neuroimaging and electrophysiological studies also confirmed changes in brain activity and structure.

### REFERENCES

1. Heilbron, M., & Chait, M. (2018). Great Expectations: Is there Evidence for Predictive Coding in Auditory Cortex? Neuroscience, 389, 54-73. https://doi.org/10.1016/j.neuroscience.2017.07.061
2. Zilles, K. (2018). Brodmann: a pioneer of human brain mapping-his impact on concepts of cortical organization. Brain, 141 (11), 3262-3278. https://doi.org/10.1093/brain/awy273
3. Breivik, H., Borchgrevink, P. C., Allen, S. M., et al. (2008). Assessment of pain. British Journal of Anaesthesia, 101 (1), 17-24. https://doi.org/10.1093/bja/aen103
4. Schaefer, M., Kühn, A., & Gärtner, M. (2022). Sensory processing sensitivity and somatosensory brain activation when feeling touch. Scientific Reports, 12, 12024. https://doi.org/10.1038/s41598-022-15497-9
5. Logothetis, N. K., & Wandell, B. A. (2008). Interpreting the BOLD signal. Annual Review of Physiology, 66, 735-769. https://doi.org/10.1146/annurev.physiol.66.082602.092845
6. Glover, G. H., Li, T. Q., & Ress, D. (2022). Image-based method for retrospective correction of physiological motion effects in fMRI: RETROICOR. Magnetic Resonance in Medicine, 44 (1), 162-167. https://doi.org/10.1002/1522-2594 (200007) 44: 1% 3C162: AID-MRM23% 3E3.0.CO; 2-E
7. Lefaucheur, J. P., et al. (2020). Evidence-based guidelines on the therapeutic use of repetitive transcranial magnetic stimulation (rTMS): An update (2014-2018). Clinical Neurophysiology, 131 (2), 474-528. https://doi.org/10.1016/j.clinph.2019.11.002
8. Volkmann, J., et al. (2010). Deep brain stimulation for the treatment of Parkinson's disease. Journal of Clinical Neurophysiology, 27 (6), 442-455. https://doi.org/10.1097/00004691-200401000-00003
9. De Ferrari, G. M., et al. (2011). Chronic vagus nerve stimulation: A new and promising therapeutic approach for chronic heart failure. European Heart Journal, 32 (7), 847-855. https://doi.org/10.1093/eurheartj/ehq391
10. McEwen, B. S., Nasca, C., & Gray, J. D. (2016). Stress effects on neuronal structure: hippocampus, amygdala, and prefrontal cortex. Neuropsychopharmacology, 41, 3-23. https://doi.org/10.1038/npp.2015.171
11. Anacker, C., & Hen, R. (2017). Adult hippocampal neurogenesis and cognitive flexibility-linking memory and mood. Nature Reviews Neuroscience, 18, 335-346. https://doi.org/10.1038/nrn.2017.45
12. Jones, L. M., Fontanini, A., Sadacca, B. F., Miller, P., & Katz, D. B. (2007). Natural stimuli evoke dynamic sequences of states in sensory cortical ensembles. Proceedings of the National Academy of Sciences, 104 (47), 18772-18777. https://doi.org/10.1073/pnas.0705546104
13. Draganski, B., Gaser, C., Kempermann, G., Kuhn, H. G., Winkler, J., Büchel, C., & May, A. (2006). Temporal and spatial dynamics of brain structure changes during extensive learning. The Journal of Neuroscience, 26 (23), 6314-6317. https://doi.org/10.1523/JNEUROSCI.4628-05.2006
14. Koo, J. W., Chaudhury, D., Han, M. H., & Nestler, E. J. (2019). Role of mesolimbic brain-derived neurotrophic factor in depression. Biological Psychiatry, 86, 738-748. https://doi.org/10.1016/j.biopsych.2019.05.020
15. Thut, G., Schyns, P. G., & Gross, J. (2011). Entrainment of perceptually relevant brain oscillations by non-invasive rhythmic stimulation of the human brain. Frontiers in Psychology, 2, 170. https://doi.org/10.3389/fpsyg.2011.00170
16. Fujioka, T., Ross, B., & Trainor, L. J. (2015). Beta-band oscillations represent auditory beat and its metrical hierarchy in perception and imagery. Journal of Neuroscience, 35 (45), 15187-15198. https://doi.org/10.1523/JNEUROSCI.2397-15.2015
17. Martorell, A. J., Paulson, A. L., Suk, H. J., et al. (2019). Multi-sensory gamma stimulation ameliorates Alzheimer's-associated pathology and improves cognition. Cell, 177 (2), 256-271.e22. https://doi.org/10.1016/j.cell.2019.02.014
18. Dogan, U., & Tahreem, F. (2023). Reduction of Porencephalic Cyst Along with the Revival of Motor Control Using the Deep Brain Sound Stimulation (DBSS) Technology as a Novel Approach. Opast Publishers, 1.0. https://doi.org/10.5281/zenodo. 12576031

1. A method for inducing brainwave entrainment, comprising: generating an audio tone using tone generator software; applying a predefined brainwave protocol to the audio tone, wherein the brainwave protocol comprises primary and secondary frequency numbers and modulation parameters; modulating the audio signals based on the primary and secondary frequency numbers using a modulation plugin; outputting the modulated audio signals to stimulate specific areas of the brain.

2. The method of claim 1, wherein the tone generator software is capable of generating a range of frequencies suitable for brainwave entrainment.

3. The method of claim 1, wherein the modulation parameters are designed to create a mix of isochronic and monaural tones to enhance the entrainment effect.

4. The method of claim 1, wherein the modulated audio signals are output through high-fidelity headphones or speakers to ensure the integrity of the signal.

5. The method of claim 1, further comprising: selecting specific brain regions to target based on the primary and secondary frequency numbers, thereby stimulating precise locations within the brain.

6. The method of claim 1, wherein primary and secondary frequency numbers are used to target specific brain regions, with primary numbers identifying major regions and secondary numbers providing detailed subregion targeting.

7. A system for brainwave entrainment, comprising: a module configured to generate an audio tone using tone generator software; a module configured to apply a brainwave protocol to the audio tone, wherein the brainwave protocol comprises primary and secondary frequency numbers and modulation parameters; a signal processor configured to modulate the audio signals based on the primary and secondary frequency numbers; an output device configured to deliver the modulated audio signals to a user.

8. A method for inducing specific physiological responses in targeted body regions, comprising: generating an audio tone using tone generator software; applying a predefined brainwave protocol to the audio tone, wherein the brainwave protocol comprises primary and secondary frequency numbers and modulation parameters; modulating the audio signals based on the primary and secondary frequency numbers using a modulation plugin; outputting the modulated audio signals to stimulate specific neural systems associated with body regions; targeting specific body locations through neural systems using decimal precision of secondary frequency numbers.
